(19) United States
(12) Patent Application Publication
(10) Pub. No.: US 2023/0349927 A1
(43) Pub. Date: Nov. 2, 2023

(54) RF biosensor and manufacturing method thereof

(71) Applicant: Industry-Academic Cooperation Foundation, YONSEI UNIVERSITY, Seoul (KR)

(72) Inventors: Seong Chan JUN, Seoul (KR); Chae Kwang IM, Ansan-si (KR); Ji Sang HA, Hanam-si (KR); Se Won PARK, Seoul (KR); Jae Kyeong LEE, Daejeon (KR); Tae Wook LEE, Seoul (KR)

(21) Appl. No.: 18/307,891

(22) Filed: Apr. 27, 2023

(30) Foreign Application Priority Data
Apr. 27, 2022 (KR) 10-2022-0051773
Apr. 21, 2023 (KR) 10-2023-0052475

(51) Int. Cl. G01N 33/68; G01N 33/543

(52) U.S. Cl. CPC G01N 33/6896; G01N 33/5438; G01N 2800/2821; G01N 2333/4709

(57) ABSTRACT

Disclosed herein are an RF biosensing system, a method of manufacturing an RF biosensor used in the same, and a method of controlling the same. The RF biosensing system allows dementia screening without any painful or uncomfortable procedure through rapid detection of biomarkers for Alzheimer's disease present in a patient's bodily fluids. In addition, the RF biosensing system is very helpful for preventing Alzheimer's disease due to the ability to detect triggers for dementia long before onset of symptoms and easily monitor the progression of Alzheimer's disease using simple and inexpensive equipment.

### FIELD

Embodiments of the present invention relate to an RF biosensing system, a method of manufacturing an RF biosensor for the same, and a method of controlling the same and, more particularly, to an RF biosensing system that allows Alzheimer's disease screening without any painful or uncomfortable procedure through rapid detection of biomarkers for Alzheimer's disease present in a patient's body fluids and is very helpful for early diagnosis and prevention of dementia due to the ability to detect triggers for Alzheimer's disease long before onset of symptoms using simple and inexpensive equipment, an RF biosensor for the same, and a method of controlling the same.

### BACKGROUND

Alzheimer's disease is the most common degenerative brain disease that causes dementia and was first reported in 1907 by a German psychiatrist named Alois Alzheimer. Alzheimer's disease develops slowly and worsens over time Alzheimer's disease typically begins with memory loss for recent events, progresses to cause various other cognitive disorders such as a speech disorder or impaired judgement, and eventually leads to loss of all daily living skills. Cognitive deterioration in Alzheimer's disease is often accompanied by psychobehavioral symptoms, such as personality changes, anxiety, depression, delusions, hallucinations, increased aggression, and sleep disturbance and, in the later stages of Alzheimer's disease, people often develop neurological disorders, such as rigidity and gait abnormalities, and physical complications, such as fecal/urine incontinence, infection, and pressure ulcers.

As a result, social and economic costs of Alzheimer's disease are rapidly increasing and various researches have been made to provide early diagnosis and prevention of the disease. However, currently reported methods for diagnosis and prevention of Alzheimer's disease are limited in their practical application due to the following problems.

First, magnetic resonance imaging (MRI) and positron emission tomography (PET) known as typical diagnostic methods for Alzheimer's disease are limited in use thereof. MRI produces images of the internal body by placing a patient's body inside a strong magnetic field, sending radio waves through the body, and measuring magnetic resonance signals, and PET produces images of the internal body by injecting a positron-emitting radioisotope tracer into a patient's body, tracking the radioisotope tracer using a positron emission tomograph, and analyzing the distribution of radioisotopes in the body. However, both MRI and PET require prolonged scan time and expensive equipment. In particular, PET can cause uncomfortable feelings for patients due to use of radioisotopes. Further, since cognitively normal patients with brain lesions rarely visit hospitals to have scans and there are a limited number of hospitals that provide MRI or PET services for patients with Alzheimer's disease, these imaging examinations are limited in use thereof.

Second, although there have been proposed diagnostic test methods for Alzheimer's disease through measurement of the concentration of amyloid-β in cerebrospinal fluid (CSF), a CSF collection procedure is time-consuming and painful to patients. In particular, CSF collection using an invasive procedure called lumbar puncture (LP) can cause the risk of spinal subdural or subarachnoid hemorrhage. Accordingly, most patients who have not yet developed symptoms will not feel the need to be tested, so they are unlikely to undergo CSF collection from their spine to be tested for Alzheimer's disease, which makes these methods unsuitable for early diagnosis and prevention of dementia.

Third, existing diagnostic test methods for Alzheimer's disease as described above have a problem in that patients do not feel the need to be tested until onset of symptoms. Although it is known that production of amyloid-β monomers and aggregation thereof into oligomeric species, which are the main triggers for Alzheimer's disease, start to appear about 20 years before onset of symptoms and preventing production and aggregation of amyloid-β helps to prevent Alzheimer's disease, these diagnostic test methods have difficulty in detection of the triggers and thus are limited in their ability to provide early diagnosis and prevention of Alzheimer's disease.

A recent study showed that Alzheimer's disease mainly develops when a small protein called amyloid-β is overproduced and deposited in the brain, causing toxic effects on brain cells. Therefore, there is an urgent need for a novel technique which can address the above problems based on this knowledge and can reduce social and economic costs of dementia by providing early diagnosis and prevention of Alzheimer's disease in a simple and rapid manner.

### SUMMARY

Embodiments of the present invention are conceived to solve such problems in the art and provide an RF biosensing system that allows dementia screening without any painful or uncomfortable procedure through rapid detection of biomarkers for Alzheimer's disease present in the patient's body fluids and is very helpful for early diagnosis and prevention of dementia due to the ability to detect triggers for dementia long before onset of symptoms using simple and inexpensive equipment, an RF biosensor for the same, and a method of controlling the same.

It will be understood that objects of the present invention are not limited to the above. The above and other objects of the present invention will become apparent to those skilled in the art from the detailed description of the following embodiments in conjunction with the accompanying drawings.

In accordance with one aspect of the present invention, there is provided an RF biosensing system for measuring degrees of production of amyloid-β and aggregation of monomeric amyloid-β into oligomeric amyloid-β using a radio frequency (RF) signal, the RF biosensing system including: an RF biosensor including a silicon substrate, a signal electrode disposed on the silicon substrate, and an interconnector electrically connected to the signal electrode to detect amyloid-β as a biomarker; and an RF analyzer electrically connected to the RF biosensor and measuring a quantitative ratio of oligomeric amyloid-β to monomeric amyloid-β using an RF signal.

In one embodiment, the interconnector may be reduced graphene oxide.

In one embodiment, the signal electrode may have a structure of a ground-signal-ground (GSG) electrode.

In one embodiment, the RF biosensing system may further include: an input probe; and an output probe, wherein the RF analyzer may receive an RF biosignal through measurement of voltage at the input probe and the output probe and may measure an S-parameter based on the RF biosignal.

In one embodiment, the RF biosensing system may further include: an artificial intelligence (AI) module adapted to build a diagnosis model through training based on the S-parameter measured by the RF analyzer, wherein the S-parameter may be a ratio of reflected/transmitted voltage measured at the output probe to reflected/transmitted voltage measured at the input probe and may include a reflection coefficient calculated using a signal reflected from the input probe and a transmission coefficient calculated using a signal reflected from the output probe, and the diagnosis model may obtain a diagnosis for Alzheimer's disease based on correlation between the reflection coefficient and the transmission coefficient.

In one embodiment, the reflection coefficient and the transmission coefficient may increase with increasing concentration of monomeric amyloid-β and oligomeric amyloid-β.

In one embodiment, the reflection coefficient may decrease and the transmission coefficient may increase with increasing ratio of monomeric amyloid-β to oligomeric amyloid-β.

In one embodiment, the signal electrode may include: a pair of central electrodes formed on an upper surface of the silicon substrate with a space therebetween and extending across the upper surface of the silicon substrate; a one-side electrode connected to both the central electrodes and extending along a periphery of the upper surface of the silicon substrate; and an opposite-side electrode connected to both the central electrodes and extending along the periphery of the upper surface of the silicon substrate to be symmetrical to the one-side electrode with respect to the pair of central electrodes.

In one embodiment, the signal electrode may further include: a pair of interconnector contact portions extending radially from distal ends of the pair of central electrodes facing each other, respectively, wherein the pair of interconnector contact portions may each include: a first interconnector contact electrode extending from one end of the central electrode in a parallel direction with respect to the central electrode; and a pair of second interconnector contact electrodes extending from the one end of the central electrode and curved with a predetermined curvature towards a distal end of the first interconnector contact electrode.

In accordance with another aspect of the present invention, a method of controlling the RF biosensing system includes the steps of: (a) introducing amyloid-β onto the RF biosensor; (b) transmitting, by the RF analyzer, an RF signal to the RF biosensor; (c) measuring, by the RF analyzer, the S-parameter based on an RF biosignal received upon passage of the RF signal through the amyloid-β; and (d) obtaining, by the AI module, a diagnosis for Alzheimer's disease through the diagnosis model trained based on the S-parameter measured by the RF analyzer.

In accordance with a further aspect of the present invention, a method of manufacturing the RF biosensor of the RF biosensing system includes the steps of: (a) forming the signal electrode on the silicon substrate; and (b) forming the interconnector detecting amyloid-β as a biomarker on the signal electrode.

In one embodiment, the step (a) may include: forming a first interconnector contact electrode extending from one end of each of the pair of central electrodes in a parallel direction with respect to the central electrode; and forming a pair of second interconnector contact electrodes extending from the one end of the central electrode and curved with a predetermined curvature towards a distal end of the first interconnector contact electrode.

In one embodiment, the step (b) may include (b1) forming graphene oxide (GO) on the signal electrode while dropping MXene thereon in a controlled ratio of the graphene oxide (GO) to MXene.

In one embodiment, the step (b) may further include (b2) baking the silicon substrate.

The RF biosensing system according to the present invention allows dementia screening without any painful or uncomfortable procedure through rapid detection of biomarkers for Alzheimer's disease present in a patient's bodily fluids and is very helpful for prevention of Alzheimer's disease due to the ability to detect triggers for dementia long before onset of symptoms and easily monitor the progression of Alzheimer's disease using simple and inexpensive equipment.

It will be understood that advantageous effects of the present invention are not limited to the above ones, and include any advantageous effects conceivable from the features disclosed in the detailed description of the present invention or the appended claims.

### DRAWINGS

The above and other aspects, features, and advantages of the present invention will become apparent from the detailed description of the following embodiments in conjunction with the accompanying drawings:

FIG. 1 is a schematic view of an RF biosensor according to one embodiment of the present invention;

FIG. 2 is a schematic view of an RF biosensing system according to one embodiment of the present invention;

FIG. 3 is a conceptual diagram illustrating a principle of measuring a reflection coefficient and a transmission coefficient based on an RF signal in the RF biosensing system according to the present invention, specifically the RF biosensor thereof;

FIG. 4 is graphs of relationships between a reflection coefficient S11 and a transmission coefficient S21 as a function of frequency for solutions having different concentrations of monomeric amyloid-β, according to one embodiment of the present invention;

FIG. 5 is graphs of relationships between the reflection coefficient S11 and the transmission coefficient S21 as a function of frequency for solutions having different concentrations of oligomeric amyloid-β, according to one embodiment of the present invention;

FIG. 6 is graphs of relationships between the reflection coefficient S11 and the transmission coefficient S21 as a function of frequency for solutions having different ratios of monomeric amyloid-β to oligomeric amyloid-β for a given total amount of monomeric and oligomeric amyloid-β;

FIG. 7 is a schematic view illustrating introduction of an interconnector onto a signal electrode in a RF biosensor manufacturing method according to one embodiment of the present invention;

FIG. 8 is a schematic view sequentially illustrating the RF biosensor manufacturing method; and

FIG. 9 is a flowchart of an RF biosensing system control method according to one embodiment of the present invention.

### DETAILED DESCRIPTION

Hereinafter, exemplary embodiments of the present invention will be described with reference to the accompanying drawings. It should be understood that the present invention may be embodied in different ways and is not limited to the following embodiments. In the drawings, portions irrelevant to the description will be omitted for clarity. Like components will be denoted by like reference numerals throughout the specification.

Throughout the specification, when an element or layer is referred to as being “on,” “connected to,” or “coupled to” another element or layer, it may be directly on, connected to, or coupled to the other element or layer or intervening elements or layers may be present. In addition, unless stated otherwise, the term “includes” should be interpreted as not excluding the presence of other components than those listed herein.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

As described above, conventional diagnostic test methods for Alzheimer's disease involve a long, painful procedure or expensive and complicated equipment and lack the ability to provide early diagnosis of Alzheimer's disease, symptoms of which take more than 20 years to manifest, resulting in significant increase in social and economic costs.

In order to solve these problems, the present invention provides: an RF biosensor for measuring the degrees of production of amyloid-β and aggregation of monomeric amyloid-β into oligomeric amyloid-β using a radio frequency (RF) signal, wherein the RF biosensor includes a silicon substrate, a signal electrode disposed on the silicon substrate, and an interconnector electrically connected to the signal electrode to detect amyloid-β as a biomarker; and a method for manufacturing an RF biosensor, which includes (a) forming a signal electrode on a silicon substrate and (b) forming an interconnector detecting amyloid-β as a biomarker on the signal electrode.

Advantageously, the RF biosensor according the present invention can rapidly detect biomarkers for Alzheimer's disease present in a patient's bodily fluids, thereby allowing Alzheimer's disease screening without any painful or uncomfortable procedure. In addition, the RF biosensor according to the present invention is able to detect triggers for Alzheimer's disease long before onset of symptoms and easily monitor the progression of Alzheimer's disease using simple and inexpensive equipment, and thus can be very helpful for prevention of Alzheimer's disease.

1. RF Biosensor 100

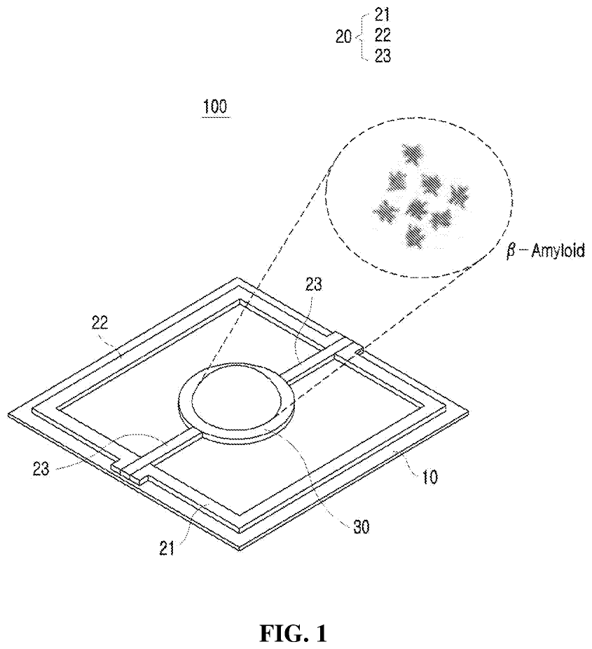

An RF biosensor according to the present invention will be described with reference to FIG. 1.

FIG. 1 is a schematic view of an RF biosensor according to one embodiment of the present invention.

Referring to FIG. 1, an RF biosensor 100 according to the present invention includes a silicon substrate 10, a signal electrode 20 disposed on the silicon substrate 10, and an interconnector 30 electrically coupled to the signal electrode 20 to detect amyloid-β as a biomarker.

The silicon substrate 10 serves as a structural material to support the RF biosensor 100 and provides a place where the signal electrode 20 is deposited, as shown in FIG. 1.

The silicon substrate 10 may be any typical silicon substrate that can be used as a structural material for a sensor capable of detecting an RF signal from a biomarker and has an appropriate thickness for preventing degradation in electrical/optical properties. Preferably, the silicon substrate 10 is a silicon wafer, more preferably a silicon wafer having an upper layer formed of silicon oxide (SiO₂) obtained by thermally oxidizing silicon, in terms of signal maximization and prevention of leakage current.

The signal electrode 20 is disposed on the silicon substrate 10, includes a pair of opposing portions spaced apart from each other to generate an RF field upon application of input/output power, and contacts an upper portion of reduced graphene oxide as the interconnector 30 described below to transmit a signal detected from a biomarker to input and output probes disposed on the silicon substrate 10 and the signal electrode 20 in the form of an electro-optical signal.

Thus, the signal electrode 20 may have a structure of a ground-signal-ground (GSG) electrode, both sides of which are grounded, as shown in FIG. 1, thereby ensuring more sensitive measurement of signal differences than when using a general single-electrode-type signal electrode.

The signal electrode 20 may be formed of any known metal consistent with the objectives of the present invention, for example, titanium (Ti), gold (Au), and chromium, preferably gold (Au) in terms of electrical conductivity and sensitivity.

Specifically, referring to FIG. 1, the signal electrode 20 includes a one-side electrode 21, an opposite-side electrode 22, and a pair of central electrodes 23.

The one-side electrode 21 is connected to both the central electrodes 23 and is formed along a periphery of an upper surface of the silicon substrate 10.

More specifically, the one-side electrode 21 is formed only on one side of the silicon substrate 10 with respect to the pair of central electrodes 23.

The opposite-side electrode 22 is connected to both the central electrodes 23 and is formed along the periphery of the silicon substrate 10 to be symmetrical to the one-side electrode 22 with respect to the pair of central electrodes 23.

More specifically, the opposite-side electrode 22 is formed on the other side of the silicon substrate 10 with respect to the pair of central electrodes 23.

Accordingly, the one-side electrode 21 and the opposite-side electrode 22 are symmetrical to each other with respect to the pair of central electrodes 23.

The pair of central electrodes 23 is formed on the upper surface of the silicon substrate 10 with a space therebetween and extends across the upper surface of the silicon substrate 10.

The interconnector 30 is formed in and near a region between the pair of central electrodes 23 facing each other.

The interconnector 30 contacts the silicon substrate 10 and the pair of central electrodes 23 of the signal electrode 20 and serves to transmit an electrical signal detected from a biomarker for Alzheimer's disease to an RF field generated by the signal electrode 20.

Although the exact pathogenesis and cause of Alzheimer's disease are not yet fully understood, a recent study showed that Alzheimer's disease mainly develops when a small protein called amyloid-β is overproduced and deposited in the brain, causing toxic effects to brain cells.

Accordingly, various research efforts are being conducted to diagnose and prevent Alzheimer's disease through quantitative measurement and analysis of amyloid-β.

However, measuring amyloid-β in blood is significantly more difficult than measuring amyloid-β in cerebrospinal fluid, which is a conventional diagnostic test method for Alzheimer's disease, since the concentration of amyloid-β in blood is 50 times lower than that in cerebrospinal fluid and other proteins than amyloid-β are approximately 1,000 times more abundant in blood than amyloid-β. Accordingly, research on amyloid-β as a biomarker for Alzheimer's disease has been limited to quantitative measurement of monomeric amyloid-β and oligomeric amyloid-β and is still far from enabling early diagnosis of Alzheimer's disease through patient-by-patient determination of a quantitative ratio between monomeric amyloid-β and oligomeric amyloid-β, that is, a degree of aggregation of amyloid-β into oligomer species. Furthermore, even though it is possible to measure the degree of aggregation of amyloid-β into oligomer species, unreliable measurement accuracy and low economic feasibility due to the necessity of expensive equipment and time-consuming analysis work limits practical use of such an approach.

The present invention can achieve accurate diagnosis and prevention of Alzheimer's disease and significant reduction in social and economic costs of the disease through early detection of Alzheimer's disease, symptoms of which take decades to manifest, in a simple and rapid manner by providing an RF biosensor including an interconnector capable of highly precise and objective measurement of the quantitative ratio between monomeric amyloid-β and oligomeric amyloid-β, which are biomarkers for Alzheimer's disease present in a patient's bodily fluids.

To this end, the RF biosensor according to the present invention may use reduced graphene oxide as an interconnector measuring the quantitative ratio between monomeric amyloid-β and oligomeric amyloid-β, which are biomarkers for Alzheimer's disease.

In general, graphene is highly promising in many applications such as electronics, energy storage and conversion devices (such as supercapacitors, batteries, fuel cells, and solar cells), and bioscience/biotechnology due to unique physical properties such as large surface area, good thermal/electrical conductivity, and high mechanical strength, and graphene oxide obtained by oxidizing graphene can be subjected to various types of chemical modification or functionalization due to a unique chemical structure thereof and thus is advantageously used in electrochemical or biomedical applications.

However, the unique chemical structure of graphene oxide can cause deterioration in electrochemical properties inherent to graphene. Accordingly, the RF biosensor according to the present invention may use reduced graphene oxide as an interconnector measuring the degree of oligomerization of amyloid-β.

In other words, in view of the fact that, despite having better electrical properties than graphene oxide, graphene oxidizes easily due to metallic properties thereof and is difficult to use in applications requiring uniformity in properties, the RF biosensor according to the present invention uses an interconnector formed of reduced graphene oxide obtained by reducing oxidized graphene under the same conditions as in oxidation to make the oxidized graphene have semiconductor characteristics, such as bandgap characteristics, and uniform properties throughout. Furthermore, since reduced graphene oxide has more functional groups than graphene and can be easily combined with biomaterials, the RF biosensor according to the present invention can sufficiently overcome the aforementioned problems by providing real-time analysis of the degree of oligomerization with high accuracy and sensitivity through use of reduced graphene oxide as an interconnector detecting amyloid-β, which is a biomarker for Alzheimer's disease, and subsequent analysis of RF signals detected from amyloid-β.

2. RF Biosensing System 200

Next, an RF biosensing system 200 according to one embodiment of the present invention will be described with reference to FIG. 1 to FIG. 6.

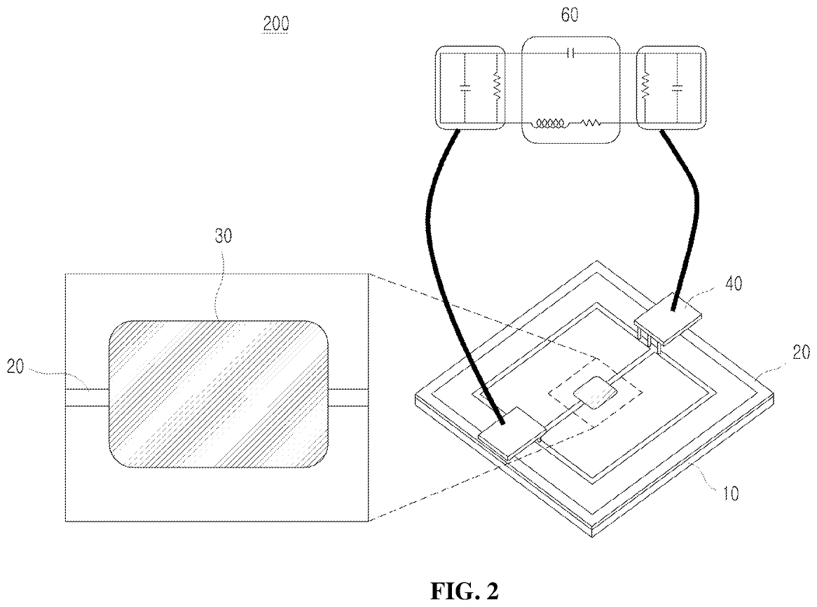

FIG. 2 is a schematic view of an RF biosensing system according to one embodiment of the present invention.

Referring to FIG. 2, the RF biosensing system 200 according to this embodiment includes an RF biosensor 100, an input probe 40, an output probe 50, and an RF analyzer 60.

More specifically, referring to FIG. 2, the present invention provides an RF biosensing system 200 including the RF biosensor 100 as described above and an RF analyzer 60 electrically connected to the RF biosensor 100 and measuring a quantitative ratio between oligomeric amyloid-β and monomeric amyloid-β based on an RF signal, wherein the RF biosensor 100 detects amyloid-β and converts the detection signal into an RF signal such that the RF analyzer 60 can analyze a degree of oligomerization of amyloid-β based on the RF signal.

The RF biosensing system 200 may further include an input probe 40 and an output probe 50, wherein each of the input probe 40 and the output probe 50 may be disposed on the silicon substrate 10 and the signal electrode (see FIG. 2) to be electrically connected to the RF analyzer 60.

Reduced graphene oxide used as the interconnector 30 of the RF biosensor 100 according to the present invention may react with a solution containing monomeric amyloid-β and oligomeric amyloid-β when a certain magnitude of voltage is flowing through the signal electrode 20, and may transmit variations in electrical properties before/after the reaction to the RF analyzer 60 described below via the signal electrode 20, such that transfer of electrons from a valence band to a conduction band can be recognized and analyzed as an electrical signal.

The RF analyzer 60 may receive an RF biosignal through measurement of voltage at the input probe 40 and the output probe 50 and may quantitatively determine a ratio between oligomeric amyloid-β and monomeric amyloid-β through measurement of an S-parameter based on the received RF biosignal.

As used herein, “S-parameter” refers to a ratio of reflected/transmitted voltage measured at the output probe 50 to reflected/transmitted voltage measured at the input probe 40 and includes a reflection coefficient S11 calculated using a signal reflected from the input probe 40 and a transmission coefficient S21 calculated using a signal reflected from the output probe 50.

The RF analyzer 60 may measure the S-parameter using input voltage and output voltage at the input probe 40 and the output probe 50.

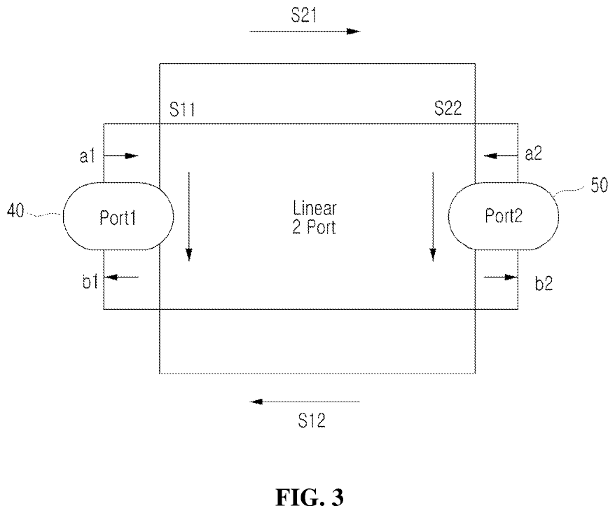

FIG. 3 is a conceptual diagram illustrating a principle of measuring the reflection coefficient and the transmission coefficient based on an RF signal in the RF biosensing system according to the present invention, specifically the RF biosensor thereof.

Specifically, referring to FIG. 3, the S-parameter is a ratio of reflected/transmitted voltage measured at the output probe 50 to reflected/transmitted voltage measured at the input probe 40, and includes a reflection coefficient (S11=b1/a1) calculated using a signal reflected from the input probe 40 and a signal reflected from the output probe 50 and a transmission coefficient (S21=b2/a1) calculated using a signal transmitted from the input probe 40 to the output probe 50 and a signal transmitted from an output port to an input port (for reference, S12=b1/a2, S22=b2/a2).

Based on the reflection coefficient S11 and the transmission coefficient S21, resistance (R), inductance (L), conductance (G), capacitance (C), impedance (Z), and propagation constant (γ) may be obtained. Specifically, R, L, G, C, Z, and y may calculated according to Equation 1:

$$[ \begin{matrix}A & B \\ C & D\end{matrix} ] = \frac{1}{2 {S}_{21}} [ \begin{matrix}( 1 - {S}_{11}^{2} + {S}_{21}^{2} ) & {Z}_{0} ( {( 1 + {S}_{11}^{2} )}^{2} - {S}_{21}^{2} ) \\ \frac{1}{{Z}_{0}} ( {( 1 - {S}_{11}^{2} )}^{2} - {S}_{21}^{2} ) & ( 1 - {S}_{11}^{2} + {S}_{21}^{2} )\end{matrix} ] \qquad ( 1 )$$

$$γ = {cosh}^{- 1} A = \sqrt{( R + j ω L ) ( G + j ω C )}$$

$$Z = \sqrt{\frac{B}{C}} = \sqrt{( R + j ω L ) / ( G + j ω C )}$$

$$R = Re \{ yZ \} L = Im \frac{\{ γ Z \}}{ω}$$

$$G = Re \{ γ / Z \} C = Im \{ γ / Z \} / ω ,$$

where Z₀ is an impedance of free space, and Re and Im are real and imaginary parts, respectively.

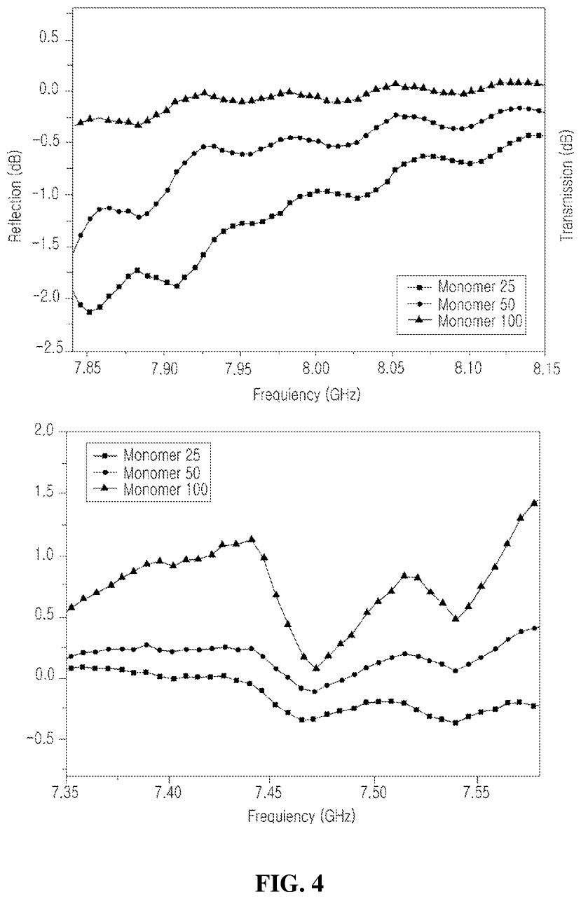

FIG. 4 is graphs of relationships between the reflection coefficient S11 and the transmission coefficient S21 as a function of frequency for solutions having different concentrations of monomeric amyloid-β, according to one embodiment of the present invention.

Referring to FIG. 4, which shows the relationships between the reflection coefficient S11 and the transmission coefficient S21 as a function of frequency, as calculated for solutions having different concentrations of monomeric amyloid-β according to Equation 1, the reflection coefficient graph (on the left) indicates that the reflection coefficient increases with increasing concentration of monomeric amyloid-β. In addition, the transmission coefficient graph (on the right) indicates that the transmission coefficient increases with increasing concentration of monomeric amyloid-β. In summary, both the reflection coefficient and the transmission coefficient increase with increasing concentration of monomeric amyloid-β.

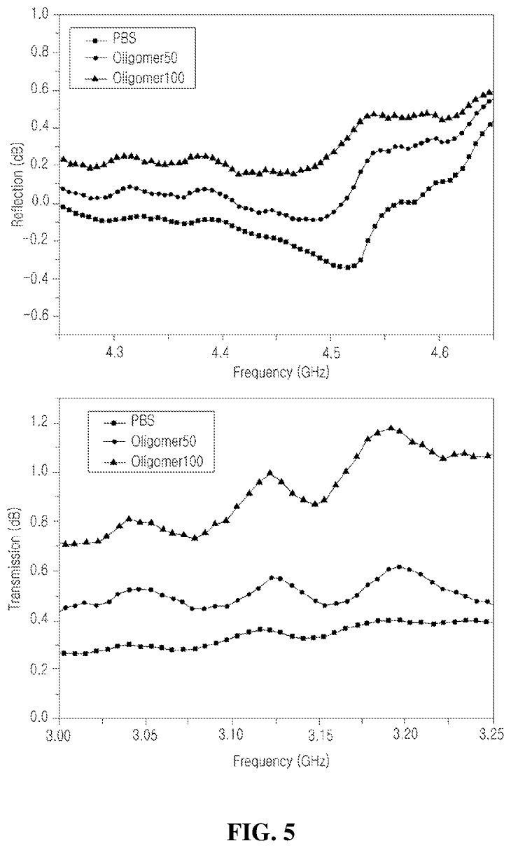

FIG. 5 is graphs of relationships between the reflection coefficient S11 and the transmission coefficient S21 as a function of frequency for solutions having different concentrations of oligomeric amyloid-β, according to one embodiment of the present invention.

Referring to FIG. 5, which shows the relationships between the reflection coefficient and the transmission coefficient as a function of frequency, as calculated for solutions placed on reduced graphene oxide and having different concentrations of oligomeric amyloid-β according to Equation 1, the reflection coefficient graph (on the left) indicates that the reflection coefficient increases with increasing concentration of oligomeric amyloid-β. In addition, the transmission coefficient graph (on the right) indicates that the transmission coefficient increases with increasing concentration of oligomeric amyloid-β. In summary, both the reflection coefficient and the transmission coefficient increase with increasing concentration of oligomeric amyloid-β.

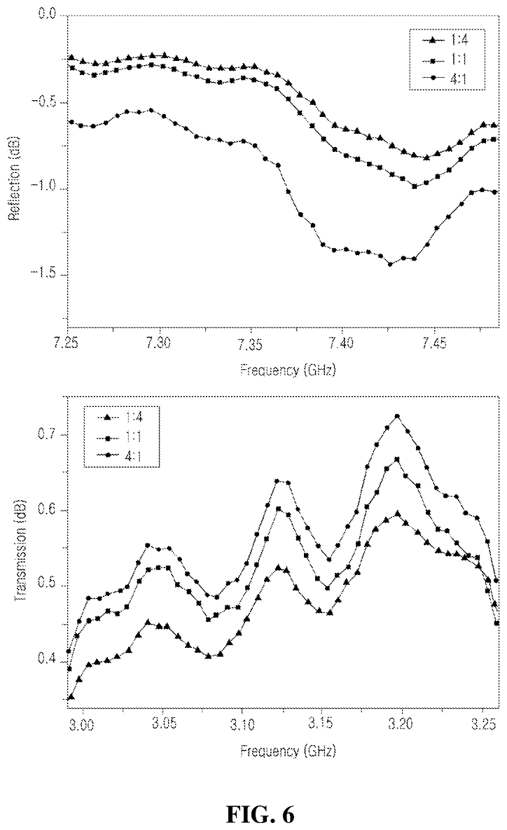

FIG. 6 is graphs of relationships between the reflection coefficient S11 and the transmission coefficient S21 as a function of frequency for solutions having different ratios of monomeric amyloid-β to oligomeric amyloid-β for a given total amount of monomeric and oligomeric amyloid-β.

Referring to FIG. 6, which shows the relationships between the reflection coefficient and the transmission coefficient as a function of frequency, as calculated for solutions placed on reduced graphene oxide and having different ratios of monomeric amyloid-β to oligomeric amyloid-β for a given total amount of monomeric and oligomeric amyloid-β, the reflection coefficient graph (on the left) indicates that the reflection coefficient decreases with increasing proportion of monomeric amyloid-β. In addition, the transmission coefficient graph (on the right) indicates that the transmission coefficient increases with increasing proportion of monomeric amyloid-β. In summary, with increasing ratio of monomeric amyloid-β to oligomeric amyloid-β, the reflection coefficient decreases, whereas the transmission coefficient increases.

In addition, the RF biosensing system 200 according to the present invention may further include an artificial intelligence (AI) module (not shown) adapted to build a diagnosis model through training based on the S-parameter (see FIG. 6) measured by the RF analyzer 60.

The diagnosis model obtains a diagnosis for Alzheimer's disease based on correlation between the reflection coefficient S11 and the transmission coefficient S21.

Accordingly, the AI module may obtain a diagnosis for Alzheimer's disease based on the correlation between the reflection coefficient S11 and the transmission coefficient S21 through the diagnosis model.

Specifically, when the ratio of monomeric amyloid-β to oligomeric amyloid-β is high, the AI module may diagnose that the probability of developing Alzheimer's disease is high based on determination that the risk of developing dementia is high.

Conversely, when the ratio of monomeric amyloid-β to oligomeric amyloid-β is low, the AI module may diagnose that the probability of developing Alzheimer's disease is low based on determination that the risk of developing dementia is low.

The RF analyzer 60 may include an interface unit, a communication unit, a display unit, a memory, and a processor, which are collectively configured to transmit an RF signal or measure the S-parameter based on the RF signal.

The interface unit may include at least one input device, for example, a keyboard, a keypad, a dome switch, a touch panel, a touch key, a mouse, and a menu button, without being limited thereto.

The communication unit may communicate with devices external to the RF analyzer 60 to receive data therefrom. For example, the communication unit may communicate with external devices via 5th generation communication (5G), long term evolution-advanced (LTE-A), long term evolution (LTE), or Wi-Fi networks.

The display unit may output display data associated with operation of the RF analyzer, wherein the data may include any data related to light intensity or wavelength, such as a screen for data input, a screen for displaying the progress of analysis, and a screen for displaying analysis results, without limitation. The display unit may include a liquid crystal display (LCD), a light emitting diode (LED) display, an organic light emitting diode (OLED) display, a micro-electromechanical system (MEMS) display, and an electronic paper display.

The memory may include a non-volatile storage that can store operating programs of the RF analyzer and can retain data (information) regardless of whether power is supplied thereto, and a volatile memory that is loaded with data to be processed by the processor and cannot retain data without power supplied thereto.

The processor may execute software, such as a program, to control one or more other components (for example, hardware or software components) of the RF analyzer, and may perform various types of data processing or computations.

As such, the RF biosensing system 200 according to the present invention can determine the degree of aggregation of amyloid-β as a biomarker for Alzheimer's disease through analysis of the S-parameter, which depends on the ratio between monomeric amyloid-β and oligomeric amyloid-β, using reduced graphene oxide as the interconnector 30 of the signal electrode 20. That is, the RF biosensing system 200 according to the present invention allows Alzheimer's disease screening without any painful or uncomfortable procedure through simple and rapid detection of biomarkers for Alzheimer's disease present in a patient's bodily fluids and is very helpful for early diagnosis and prevention off dementia due to the ability to detect triggers for dementia long before onset of symptoms using simple and inexpensive equipment.

3. RF Biosensor Manufacturing Method

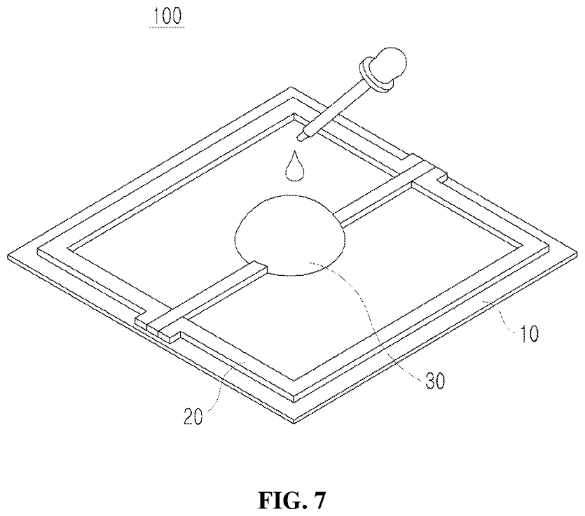

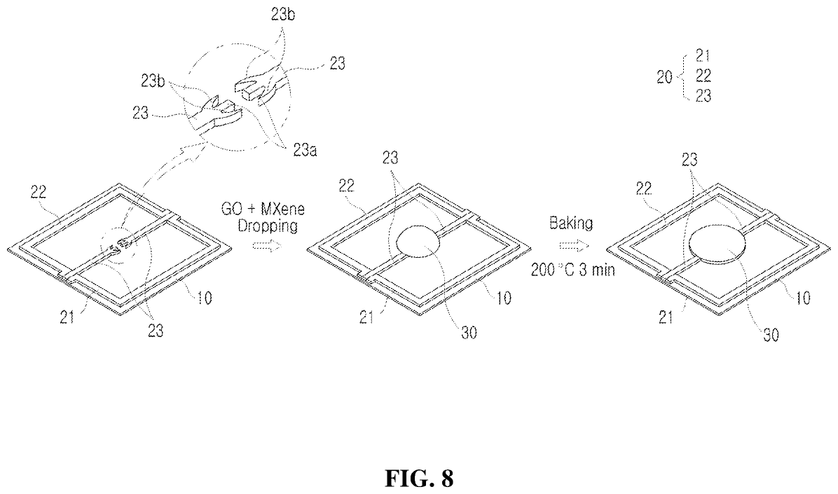

Next, an RF biosensor manufacturing method according to one embodiment of the present invention will be described with reference to FIG. 7 and FIG. 8.

In the following, in order to avoid redundancy, description of the same technical features as those described above relating to the RF biosensor 100 and the RF biosensing system 200 will be omitted.

FIG. 7 is a schematic view illustrating introduction of an interconnector onto a signal electrode in the RF biosensor manufacturing method according to this embodiment. FIG. 8 is a schematic view sequentially illustrating the RF biosensor manufacturing method.

Referring to FIG. 7 and FIG. 8, the RF biosensor manufacturing method according to the present invention is a method of manufacturing the RF biosensor of the RF biosensing system as described above, and includes: (a) forming the signal electrode 20 on the silicon substrate 10; and (b) forming the interconnector 30 detecting amyloid-β as a biomarker on the signal electrode 20.

Step (a) includes: forming a first interconnector contact electrode 23a extending from one end of each of the pair of central electrodes 23 in a parallel direction with respect to the central electrode 23; and forming a pair of second interconnector contact electrodes 23b extending from the one end of the central electrode 23 and curved with a predetermined curvature towards a distal end of the first interconnector contact electrode 23a.

Specifically, step (a) includes forming a pair of interconnector contact portions extending radially from distal ends of the pair of central electrodes 23 facing each other, respectively.

More specifically, each of the pair of interconnector contact portions includes: a first interconnector contact electrode 23a extending from one end of the central electrode 23 in a parallel direction with respect to the central electrode 23; and a pair of second interconnector contact electrodes 23b each extending from the one end of the central electrode 23 and curved with a predetermined curvature toward a distal end of the first interconnector contact electrode 23a.

That is, the first interconnector contact electrode 23a and the pair of second interconnector contact electrodes 23b formed at the distal end of each of the pair of central electrodes 23 may collectively form a fork shape or a trident shape, as shown on the left side of FIG. 8, without being limited thereto.

Thus, this three-pronged shape allows the first interconnector contact electrode 23a and the pair of second interconnector contact electrodes 23b to softly contact the interconnector 30 to ensure smooth application and measurement of an RF field (alternating current that generates an electromagnetic field for wireless broadcasting or communication by transmitting current through an antenna when passing through the antenna) without frequency scattering, thereby improving the concentration of the RF field and ultimately improving the performance of the RF sensor.

In addition, the structural shape of the first and second interconnector contact electrodes 23a, 23b is applied equally to the RF biosensor 100 and RF biosensing system 200 described above.

Conventional diagnostic test equipment for Alzheimer's disease generally requires complex design and manufacturing processes, which significantly limits mass production and utilization in various industries. The RF biosensor manufacturing method according to present invention enables mass production of an RF biosensor capable of simple and rapid diagnosis for Alzheimer's disease through simple two steps (steps a and b) described above, thereby significantly reducing social and economic costs of Alzheimer's disease.

To this end, in step (a), the signal electrode 20 is formed on the silicon substrate 10.

Forming the signal electrode on the silicon substrate may be performed by any conventional electrode formation method known in the art, so long as the method does not affect sensing accuracy.

In a preferred example, a photolithography process may be used to form the signal electrode on the silicon substrate. The photolithography process may include applying a photoresist to the silicon substrate, using a photomask patterned with the signal electrode to remove the photoresist according to the pattern, and forming the signal electrode using electron beam deposition. The photolithography process may be advantageous in terms of mass production and uniform quality between different substrates.

The signal electrode may have a GSG electrode type signal electrode and may be formed of any typical known material consistent with the objectives of the present invention. For example, the signal electrode may be formed of titanium (Ti), gold (Au), or chromium (Cr), preferably gold (Au) in terms of electrical conductivity and sensitivity.

Prior to forming the signal electrode on the silicon substrate, the silicon substrate may be subjected to a process in which an upper layer thereof having a thickness of 100 μm to 1,000 μm and an area of 3 mm to 30 mm is thermally oxidized into a silicon oxide (SiO₂) layer. Here, the thermal oxidation process may include any typical thermal oxidation process consistent with the objectives of the present invention. For example, the upper layer of the silicon substrate may be oxidized into a silicon oxide (SiO2) layer by thermal oxidation at 800° C. to 1,200° C. for about 10 to 50 minutes. Advantageously, the silicon oxide substrate thus formed can further reduce leakage current and maximize RF signals, as compared with typical silicon substrates.

In step (b), the interconnector 30 detecting amyloid-β as a biomarker is formed on the signal electrode.

Referring to FIG. 8, step (b) includes (b1) forming graphene oxide (GO) on the signal electrode while dropping MXene thereon in a controlled ratio of graphene oxide (GO) to MXene.

Step (b) further includes (b2) baking the silicon substrate.

That is, since the RF biosensor according to the present invention uses reduced graphene oxide as an interconnector measuring the degree of oligomerization of amyloid-β, as described above, step (b) may include (b1) forming graphene oxide (GO) on the signal electrode while dropping MXene thereon in a controlled ratio of the graphene oxide (GO) to MXene and (b2) baking the silicon substrate, such that the reduced graphene oxide is formed on the signal electrode through thermal reduction of the graphene oxide (GO) and MXene.

Forming graphene oxide on the signal electrode in step (b1) may be performed by any typical graphene oxide formation method known in the art. For example, referring to FIG. 7, using a dropper, graphene oxide may be injected as precisely as desired into a region between the pair of central electrodes of the GSG signal electrode to be brought into contact with both the central electrodes.

In step (b1), the graphene oxide (GO) and MXene as a TMD material are combined in a predetermined ratio.

If an excess of MXene is mixed with the graphene oxide (GO), this can cause reduction in conductivity. Accordingly, there is a need to adjust the ratio of the graphene oxide (GO) to MXene within an appropriate range.

Mixing of the graphene oxide (GO) and MXene can significantly increase the performance of the RF biosensor since more bio-functional groups are generated when the graphene oxide (GO) and MXene are combined.

Reducing the graphene oxide formed on the signal electrode in step (b2) may be performed by any typical graphene oxide reduction method consistent with the objectives of the present invention. For example, referring to FIG. 8, the graphene oxide may be thermally reduced into the reduced graphene oxide by baking the entirety of the silicon substrate including the graphene oxide at about 100° C. to 300° C. for 1 to 10 minutes.

Next, the present invention will be described in more detail with reference to examples. However, it should be noted that these examples are provided for illustration only and should not be construed in any way as limiting the invention.

### Example 1

A silicon substrate with a thickness of about 675 μm and an area of 9*9 mm was subjected to thermal oxidation at about 1,000° C., thereby fabricating a silicon oxide (SiO2) substrate.

Thereafter, PMMA 950 k A6 was applied to the silicon oxide substrate by spin coating at 3,000 rpm for 30 seconds using a spin coater. Thereafter, the silicon oxide substrate was irradiated with electron beams according to the shape of a GSG electrode using a field emission scanning electron microscope (FE-SEM). Here, irradiation with electron beams served to modify PMMA to be removable by a developer. Thereafter, the substrate was dipped in a developer, such that the entire surface of the substrate was covered with a PMMA layer, excluding a region irradiated with electron beams (that is, a GSG electrode-shaped region).

Thereafter, a 5 nm thick chromium layer and a 50 nm thick gold layer were sequentially deposited on the substrate to form a signal electrode. Here, the chromium layer served as an adhesion layer facilitating deposition of the gold layer on the silicon oxide substrate. Through this process, a developed region had metals deposited directly on the silicon substrate and an undeveloped region had metals deposited on the PMMA layer. Thereafter, the substrate was dipped in acetone to remove the PMMA layer, such that the metals deposited on the PMMA layer were also removed and the metals deposited on the silicon substrate remained unremoved.

Thereafter, 10 μl of graphene oxide was dropped onto the signal electrode using a dropper, followed by baking at 200° C. for 3 minutes to form reduced graphene oxide, thereby obtaining the RF biosensor according to the present invention.

### Experimental Example 1: Measurement of Reflection Coefficient S11 and Transmission Coefficient S21 Under Varying Concentrations of Monomeric Amyloid-β

Using the RF biosensor manufactured in Example 1 in conjunction with a CPX-VF chamber and probe station (Lakeshore Cryotronics) and an E8364B PNA network analyzer (Keysight/Agilent Technologies), the reflection coefficient S11 and the transmission coefficient S21 were measured under varying concentrations of monomeric amyloid-β (25 pg/ml, 50 pg/ml, 100 pg/ml (see FIG. 4)).

Referring to FIG. 4, the reflection coefficient graph (on the left) indicates that the reflection coefficient increases with increasing concentration of monomeric amyloid-β. In addition, the transmission coefficient graph (on the right) indicates that the transmission coefficient increases with increasing concentration of monomeric amyloid-β. In summary, both the reflection coefficient and the transmission coefficient increase with increasing concentration of monomeric amyloid-β.

### Experimental Example 2: Measurement of Reflection Coefficient S11 and Transmission Coefficient S21 Under Varying Concentrations of Oligomeric Amyloid-β

Using the RF biosensor manufactured in Example 1 in conjunction with a CPX-VF chamber and probe station (Lakeshore Cryotronics) and an E8364B PNA network analyzer (Keysight/Agilent Technologies), the reflection coefficient S11 and the transmission coefficient S21 were measured under varying concentrations of oligomeric amyloid-β (PBS solution, 50 pg/ml, 100 pg/ml (see FIG. 6)).

Referring to FIG. 5, the reflection coefficient graph (on the left) indicates that the reflection coefficient increases with increasing concentration of oligomeric amyloid-β. In addition, the transmission coefficient graph (on the right) indicates that the transmission coefficient increases with increasing concentration of oligomeric amyloid-β. In summary, both the reflection coefficient and the transmission coefficient increase with increasing concentration of oligomeric amyloid-β.

### Experimental Example 3: Measurement of Reflection Coefficient S11 and Transmission Coefficient S21 Under Varying Ratios of Monomeric Amyloid-β to Oligomeric Amyloid-β

Using the RF biosensor manufactured in Example 1 in conjunction with a CPX-VF chamber and probe station (Lakeshore Cryotronics) and an E8364B PNA network analyzer (Keysight/Agilent Technologies), the reflection coefficient S11 and the transmission coefficient S21 were measured under varying ratios of monomeric amyloid-β to oligomeric amyloid-β (1:4, 1:1, 4:1 (see FIG. 6)) for a given total amount of monomeric amyloid-β and oligomeric amyloid-β.

Referring to FIG. 6, the reflection coefficient graph (on the left) shows a peak around 7.42 GHz, which indicates that the reflection coefficient decreases with increasing proportion of monomeric amyloid-β. In addition, the transmission coefficient graph (on the right) shows peaks at 3.05 GHz, 3.12 GHz, and 3.2 GHz, which indicates that the transmission coefficient increases with increasing proportion of monomeric amyloid-β. In summary, with increasing ratio of monomeric amyloid-β to oligomeric amyloid-β, the reflectance coefficient decreases, whereas the transmission coefficient increases.

4. RF Biosensing System Control Method

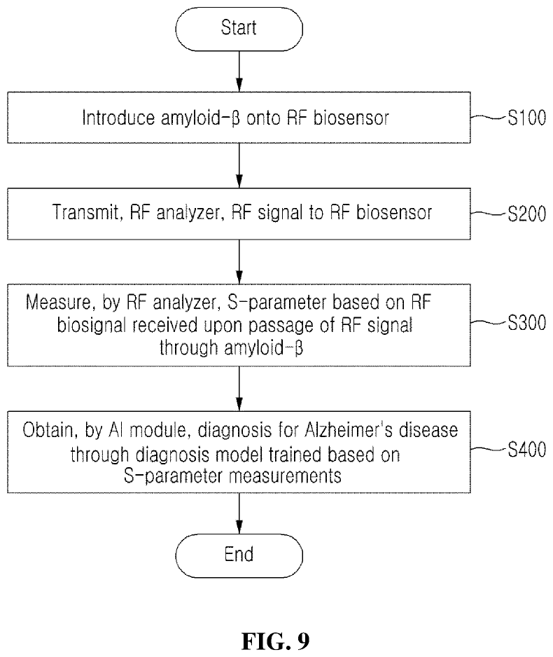

Next, an RF biosensing system control method according to one embodiment of the present invention will be described with reference to FIG. 1, FIG. 2, and FIG. 9.

FIG. 9 is a flowchart of an RF biosensing system control method according to one embodiment of the present invention.

Referring to FIG. 9, the RF biosensing system control method according to this embodiment includes (a) introducing amyloid-β onto to the RF biosensor 100, (b) transmitting, by the RF analyzer 60, an RF signal to the RF biosensor 100, (c) measuring, by the RF analyzer 60, an S-parameter based on an RF biosignal received upon passage of the RF signal through the amyloid-β, and (d) obtaining, by the AI module, a diagnosis for Alzheimer's disease through a diagnosis model trained based on the S-parameter measured by the RF analyzer 60.

In step (a), amyloid-β may be introduced onto the interconnector 30 of the RF biosensor 100 using an appropriate device (not shown).

In step (b), the RF analyzer 60 generates an RF signal and transmits the generated RF signal to the input probe 40 electrically connected thereto.

Step (c) includes (c1) receiving, by the signal electrode 20, the RF signal transmitted from the input probe 40, (c2) allowing, by the signal electrode 20, the RF signal to pass through the amyloid-β, (c3) transmitting, by the signal electrode 20, an RF biosignal received upon passage of the RF signal through the amyloid-β to the output probe 50, and (c4) measuring, by the RF analyzer 60, the S-parameter based on the RF biosignal transmitted from the output probe 50.

In step (d), an S-parameter signal related to the S-parameter includes a reflection signal and a transmitted signal.

Specifically, step (d) includes (d1) receiving, by the AI module, an S-parameter signal related to the S-parameter, (d2), building, by AI module, a diagnosis model through training based on the S-parameter measured by the RF analyzer 60, and (d3) applying, by the AI module, a newly measured S-parameter signal to the diagnosis module to obtain a diagnosis for Alzheimer's disease.

As described above, the RF biosensing system according to the present invention allows Alzheimer's disease screening without any painful or uncomfortable procedure through rapid detection of biomarkers for Alzheimer's disease present in a patient's bodily fluids. In addition, the RF biosensing system according to the present invention is very helpful for preventing Alzheimer's disease due to the ability to detect triggers for Alzheimer's disease long before onset of symptoms and easily monitor the progression of Alzheimer's disease using simple and inexpensive equipment.

Although some embodiments have been described herein, it should be understood that these embodiments are provided for illustration only and are not to be construed in any way as limiting the present invention, and that various modifications, changes, alterations, and equivalent embodiments can be made by those skilled in the art without departing from the spirit and scope of the invention. For example, components described as implemented separately may also be implemented in combined form, and vice versa.

The scope of the present invention is indicated by the following claims and all changes or modifications derived from the meaning and scope of the claims and equivalents thereto should be construed as being within the scope of the present invention.

1. An RF biosensing system for measuring degrees of production of amyloid-β and aggregation of monomeric amyloid-β into oligomeric amyloid-β using a radio frequency (RF) signal, the RF biosensing system comprising:
an RF biosensor comprising a silicon substrate, a signal electrode disposed on the silicon substrate, and an interconnector electrically connected to the signal electrode to detect amyloid-β as a biomarker; and
an RF analyzer electrically connected to the RF biosensor and measuring a quantitative ratio of monomeric amyloid-β to oligomeric amyloid-β using an RF signal.

2. The RF biosensing system according to claim 1, wherein the interconnector is reduced graphene oxide.

3. The RF biosensing system according to claim 1, wherein the signal electrode includes a ground-signal-ground (GSG) electrode.

4. The RF biosensing system according to claim 1, further comprising:
an input probe; and an output probe,
wherein the RF analyzer is configured to receive an RF biosignal through measurement of voltage at the input probe and the output probe and measure an S-parameter based on the RF biosignal.

5. The RF biosensing system according to claim 4, further comprising:
an artificial intelligence (AI) module configured to build a diagnosis model through training based on the S-parameter measured by the RF analyzer,
wherein the S-parameter is a ratio of reflected/transmitted voltage measured at the output probe to reflected/transmitted voltage measured at the input probe and comprises a reflection coefficient calculated using a signal reflected from the input probe and a transmission coefficient calculated using a signal transmitted from the output probe, and
the diagnosis model obtains a diagnosis for Alzheimer's disease based on correlation between the reflection coefficient and the transmission coefficient.

6. The RF biosensing system according to claim 1, wherein the reflection coefficient and the transmission coefficient increase with increasing concentration of monomeric amyloid-β and oligomeric amyloid-β.

7. The RF biosensing system according to claim 1, wherein the reflection coefficient decreases and the transmission coefficient increases with increasing ratio of monomeric amyloid-β to oligomeric amyloid-β.

8. The RF biosensing system according to claim 1, wherein the signal electrode comprises:
a pair of central electrodes formed on an upper surface of the silicon substrate with a space therebetween and extending across the upper surface of the silicon substrate;
a one-side electrode connected to both the central electrodes and extending along a periphery of the upper surface of the silicon substrate; and
an opposite-side electrode connected to both the central electrodes and extending along the periphery of the upper surface of the silicon substrate to be symmetrical to the one-side electrode with respect to the pair of central electrodes.

9. The RF biosensing system according to claim 8, wherein the signal electrode further comprises:
a pair of interconnector contact portions extending radially from distal ends of the pair of central electrodes facing each other, respectively, each of the pair of interconnector contact portions comprising:
a first interconnector contact electrode extending from one end of a central electrode in a parallel direction with respect to the central electrode; and
a pair of second interconnector contact electrodes extending from the one end of the central electrode and curved with a predetermined curvature towards a distal end of the first interconnector contact electrode.

10. A method of controlling an RF biosensing system, comprising:
(a) introducing amyloid-β onto an RF biosensor;
(b) transmitting, by an RF analyzer, an RF signal to the RF biosensor;
(c) measuring, by the RF analyzer, an S-parameter based on an RF biosignal received upon passage of the RF signal through the amyloid-β; and
(d) obtaining, by an artificial intelligence (AI) module, a diagnosis for Alzheimer's disease through a diagnosis model trained based on the S-parameter measured by the RF analyzer.

11. A method of manufacturing the RF biosensor of the RF biosensing system according to claim 8, the method comprising:
(a) forming the signal electrode on the silicon substrate; and
(b) forming the interconnector detecting amyloid-β as a biomarker on the signal electrode.

12. The method according to claim 11, wherein the step (a) comprises:
forming a first interconnector contact electrode extending from one end of each of the pair of central electrodes in a parallel direction with respect to the central electrode; and
forming a pair of second interconnector contact electrodes extending from the one end of the central electrode and curved with a predetermined curvature towards a distal end of the first interconnector contact electrode.

13. The method according to claim 11, wherein the step (b) comprises (b1) forming graphene oxide (GO) on the signal electrode while dropping MXene thereon in a controlled ratio of the graphene oxide (GO) to MXene.

14. The method according to claim 13, wherein the step (b) further comprises (b2) baking the silicon substrate.
